(19) United States
(12) Patent Application Publication
(10) Pub. No.: US 2014/0065701 A1
(43) Pub. Date: Mar. 6, 2014

(54) METHOD FOR HARVESTING MICROALGAE AND DEVICE FOR IMPLEMENTING SAID METHOD

(71) Applicant: Edouard Kabakian, Gaillac (FR)

(72) Inventor: Edouard Kabakian, Gaillac (FR)

(21) Appl. No.: 14/115,142

(22) PCT Filed: Mar. 27, 2012
(86) PCT No.: PCT/FR2012/050640

§ 371 Date: Nov. 1, 2013

(30) Foreign Application Priority Data
May 2, 2011 (FR) 1101349

(51) Int. Cl. C12N 1/12

(52) U.S. Cl. CPC C12N 1/12
USPC 435/257.1; 435/308.1

(57) ABSTRACT

Method for harvesting microalgae in a culture medium, includes passing the medium through a set of consecutive concentrators; each including a passive filtering unit separating the inner space of the concentrator into an upper and lower volume; the upper volume having an inlet (16) and an outlet (18) arranged at a lower position with respect to the inlet for extracting a more concentrated culture medium from the upper volume; the lower volume having an outlet (32) for extracting a liquid from the lower volume; the inlet (16) of a downstream concentrator being connected to the outlet (18) of the upper volume of the upstream concentrator; and harvesting a concentrated culture medium at the outlet (18) of the upper volume of a concentrator. Preferably, the inlet of the upper volume of an upstream concentrator is located at a greater height than the inlet of the upper volume of the downstream concentrator.

The present invention relates to a method for harvesting microalgae and a device for implementing such a method.

Microalgae and cyanobacteria are aquatic organisms ranging in size from microns to hundreds of microns, using light as an energy source to bind carbon dioxide (CO₂). Like terrestrial plants, microalgae and cyanobacteria can accumulate absorbed carbon in the form of lipids, so that their use can be envisaged to produce biofuels. Such a use is all the more promising since microalgae and cyanobacteria show a very high photosynthetic efficiency and cell growth rate (one to several tens times higher than those of terrestrial oilseeds such as rapeseed, sunflower, . . . ) and the fraction of directly usable biomass is maximum (conversely, terrestrial plants divert some of the absorbed carbon towards lignocellulosic molecules, which are more difficult or impossible to exploit).

There are currently two main ways to produce microalgae and cyanobacteria: open air culture in ponds of the “racetrack” type, and cultures in a closed transparent enclosure known as a photobioreactor. Open cultures offer lower yields, require a large water intake to compensate for evaporation and are susceptible to contamination. Photobioreactors can offset a higher cost with high productivity through greater control of the conditions of access to food resources, light exposure, and CO₂ transfer from the gas phase to the liquid phase. Such a photobioreactor is for example disclosed in FR 2,946,362.

Through these production means, culture media in which microalgae are present at a concentration of a few grams of dry matter per liter of culture medium are obtained. In order to be able to exploit microalgae, for example, in the production of biofuels, or other products (food products, cosmetics, . . . ), it is necessary to obtain a medium having a much higher concentration.

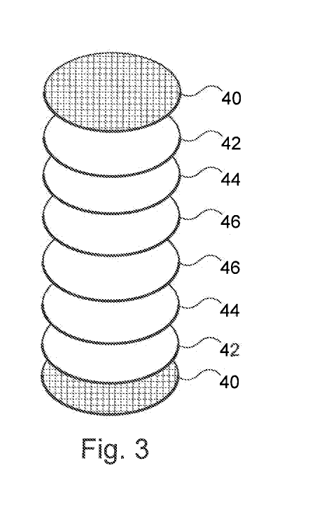

Prior art techniques for concentrating the dry matter content rely, for example, on centrifugation of the culture medium. These techniques are generally used after a sedimentation step to increase centrifugation efficiency. A device such as that shown in FIGS. 2 and 3 of EP 1671704 is used for example to remove water from a concentrate obtained after a first microalgae preconcentration step using sedimentation.

These various methods are most often carried out in several consecutive stages and result in a relatively high energy consumption. When the final balance is considered, particularly for biofuels, the proportion of energy involved in the microalgae concentration step is relatively high.

US2009/0162919 discloses a method for concentrating microalgae in an aqueous environment, with this method comprising: contacting microalgae with an inorganic flocculant present at a concentration that is less than 10% of dry matter, and separating the flocs of obtained microalgae from the aqueous environment, thereby concentrating the microalgae into a slurry with a biomass density of at least 1%. The proposed method is limited to unicellular microalgae with an average diameter of less than 20 μm. Furthermore, the addition of a foreign product (flocculant) to the culture medium may limit the usability of the resulting slurry (for example, it is known that the application of aluminum-containing cosmetics to the skin presents a risk) and increases the cost of the method. In addition, the use of the recommended flocculants (in particular, aluminum or iron-based flocculants) is not environmentally neutral.

An object of the present invention is therefore to provide a new method for obtaining a high concentration of microalgae in a culture medium without the addition of foreign solid matter to the culture medium, preferably with as low an energy consumption as possible. Another object of the invention is to provide a concentrating and harvesting method wherein the microalgae (living organisms) do not undergo any shock or lethal treatment. Moreover, this method can, advantageously, provide a concentrated microalgae medium in a continuous manner. In addition, the obtained concentration will advantageously allow for direct use in the production of biofuels or other microalgae-derived products.

For that purpose, the present invention provides a method for harvesting microalgae found in a culture medium.

According to the invention, the method is characterized in that:
the culture medium passes through a set of consecutive tanks, referred to as concentrators, thereby defining a series of upstream and downstream concentrators,
each concentrator has, on the one hand, a casing which defines an inner space and, on the other hand, a passive filtering unit which separates said inner space into an upper volume and a lower volume,
the upper volume comprises an inlet for introducing a culture medium into said upper volume and an outlet arranged at a lower position relative to the inlet for extracting from the upper volume a culture medium having a higher microalgae concentration,
the lower volume comprises an outlet for extracting a liquid from the lower volume,
in other words, each concentrator has, on the one hand, an inlet, and on the other hand, two outlets separated by a passive filtering unit; the presence of two outlets enables the force of gravity to act through two resultants; the culture medium which enters a concentrator is thus divided within the latter;
the inlet of the upper volume of a downstream concentrator is connected to the outlet of the upper volume of the upstream concentrator,
a concentrated culture medium is harvested at the outlet of the upper volume of a concentrator. For example, harvesting is carried out at the outlet of the last (most downstream) concentrator of the whole system. Depending on the desired concentration, in particular according to the intended use of the culture medium, harvesting may however be done further upstream.

It should be noted that the terms upstream and downstream as used herein to describe the concentrators are relative terms: each concentrator (except the first and last concentrators in the set) can be termed in turn as the downstream concentrator or upstream concentrator, according to whether the observed pair of concentrators is formed by said concentrator and the previous one, or by said concentrator and the next one in the series of concentrators (in the direction along which the culture medium flows).

This method allows a series of concentrations of culture medium to be carried out. For gravity-only operation, and thus to reduce the energy consumed for harvesting microalgae, the inlet of the upper volume of an upstream concentrator is advantageously located at a greater height than the inlet of the upper volume of the downstream concentrator.

To regulate the pressure within a concentrator and also promote drainage of microalgae towards the outlet of the upper volume of the concentrator, carbon dioxide can be injected into the lower volume of the relevant concentrator (preferably at the level of each concentrator). The injection of carbon dioxide has a third advantage, which is to prevent clogging of the passive filtering unit and any sedimentation of microalgae in the upper volume of the concentrator.

The present invention also relates to a device for harvesting microalgae, characterized in that it comprises a set of consecutive tanks, referred to as concentrators, thereby defining a series of upstream and downstream concentrators, in that each concentrator has, on the one hand, a casing which defines an inner space and, on the other hand, a passive filtering unit which separates said inner space into an upper volume and a lower volume, in that the upper volume comprises an inlet for introducing a culture medium into said upper volume and an outlet arranged at a lower position relative to the inlet for extracting a culture medium having a higher microalgae concentration from the upper volume, in that the lower volume comprises an outlet for extracting a liquid from the lower volume, and in that the inlet of the upper volume of a downstream concentrator is connected to the outlet of the upper volume of the upstream concentrator. Such a device allows a method according to the present invention to be implemented.

As mentioned above, to allow the device to operate by gravity only, the inlet of the upper volume of an upstream concentrator is advantageously located at a greater height than the inlet of the upper volume of the downstream concentrator.

In a device according to the present invention, the lower volume of at least one concentrator preferably includes means for injecting a pressurized gas into said lower volume.

For easier implementation, all concentrators are placed in the same plane. However, it is for example possible to provide consecutive stepped planes to accommodate the consecutive concentrators of a device according to the invention. In the case where all concentrators are placed in the same plane, it is then also possible for all passive filter units of the device to be arranged at the same height, that is, at the same height relative to said plane on which the concentrators rest.

A device according to the present invention is preferably sealed to prevent contamination of the algal medium. However, since oxygen is released by the algal medium, the upper volume of at least one concentrator advantageously has in its upper portion a membrane which is semi-permeable to oxygen, allowing oxygen to exit the concentrator. Alternatively or in combination a membrane which is semi-permeable to hydrogen may also be present in the upper portion of the concentrator, if the medium equally rejects hydrogen.

Another object of the present invention is also to provide a novel passive filtering unit, which can advantageously be used in a concentrator according to the present invention or in other applications. Such a passive filtering unit comprises two wire meshes between which filter membranes are located, the porosity of which decreases from one wire mesh towards the center of the passive filtering unit.

Features and advantages of the present invention will become more apparent from the following description with reference to the accompanying drawings, in which:

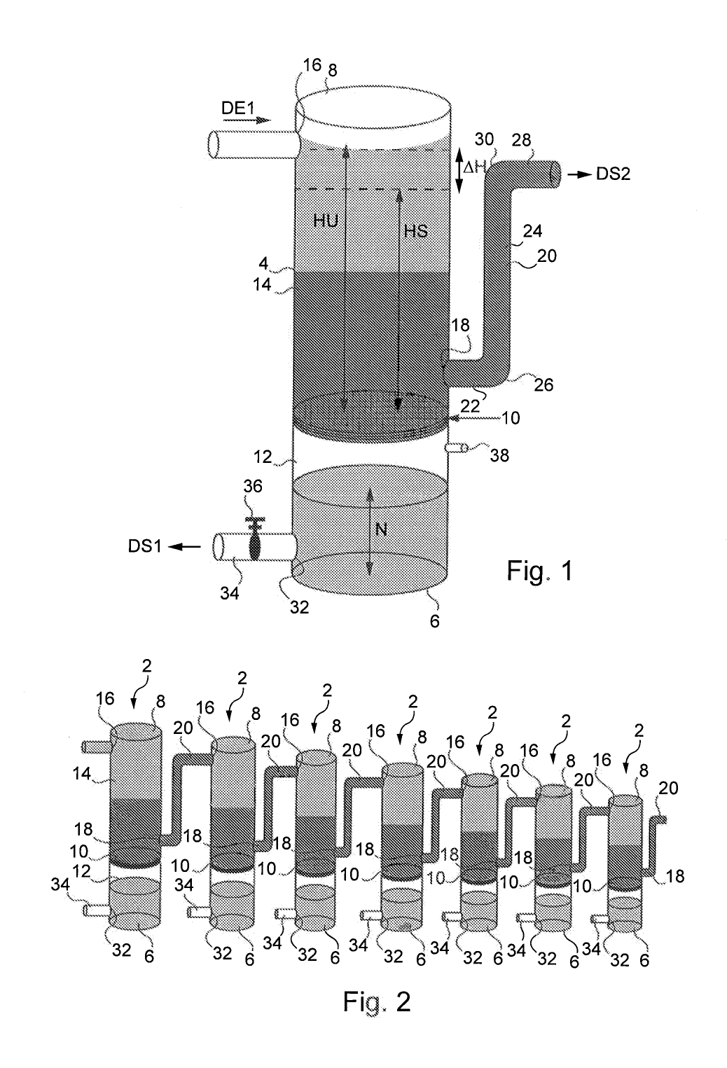

FIG. 1 shows an embodiment of a concentrator which may be used in a device for harvesting microalgae according to the present invention,

FIG. 2 shows a device for harvesting microalgae according to the present invention, which employs seven concentrators as shown in FIG. 1, and

FIG. 3 illustrates a passive filtering unit used in a concentrator of a harvesting device according to the present invention.

FIG. 1 illustrates a concentrator 2 used for implementing the present invention. As illustrated in FIG. 2, several concentrators of the type shown in FIG. 1 are combined with each other to provide a device for harvesting microalgae and to implement a method for harvesting microalgae according to the present invention.

Concentrator 2 of FIG. 1 has a circular cylindrical overall shape corresponding to a preferred embodiment of the present invention. It has an outer casing with a sidewall 4, a bottom 6 and a cover 8. In this embodiment, sidewall 4 is circularly cylindrical in shape. The bottom 6 and cover 8 are in turn disc shaped, with the disc radius corresponding to the radius of the circular cylinder of sidewall 4. Sidewall 4 and bottom 6 are for example made of steel, preferably stainless steel. Other materials and other shapes can of course be considered here. A transparent sidewall 4 may for example advantageously be provided so that the microalgae present in the culture medium can benefit from ambient light, and can grow.

Concentrator 2 is designed to be placed on a flat horizontal surface, bottom 6 then coming into contact with the ground. The following description assumes that each concentrator 2 is in such a position whenever the up/down and upper/lower directions are mentioned.

A passive filtering unit 10, which is shown in greater detail in FIG. 3 and will be described in further detail below, is located inside the casing of the concentrator. The passive filtering unit 10 has the overall shape of a disc and is arranged in the concentrator 2, parallel to bottom 6 and cover 8, between these, so as to define, in the casing of the concentrator, a partition which defines a lower volume 12 and an upper volume 14.

Here, the provision of a two-portion sidewall 4 can be envisaged: a lower portion extending from the bottom 6 up to the passive filtering unit 10 and an upper portion extending from the passive filtering unit 10 to cover 8. The passive filtering unit 10 then rests, for example, on a base (not shown) machined in the upper edge of the lower portion of sidewall 4 in order to accommodate the passive filtering unit 10. The upper portion of sidewall 4 can then, for example, be arranged so as to maintain the passive filtering unit 10 so that it rests on the lower portion of sidewall 4. Sidewall 4 can also be formed in one piece, in which case means can be provided in the interior thereof to accommodate the passive filtering unit 10. In all cases, a sealed connection is preferably provided between the passive filtering unit 10 and sidewall 4, or between sidewall 4 and a support provided for accommodating and housing the passive filtering unit 10.

Since the passive filtering unit 10 can be subjected to considerable forces, depending in particular on the pressure exerted on it by the culture medium in the upper volume 14 and also depending on the surface area of the passive filtering unit 10, a variety of means can be provided to support this passive filtering unit 10, not only at its periphery. Cross struts can support the passive filtering unit 10 or a support which rests on the bottom 6 of the concentrator can also be provided to hold the passive filtering unit 10. A number of solutions can be considered here for supporting this passive filtering unit. The skilled person will choose a solution adapted to the mechanical stresses to which the passive filtering unit 10 will be subjected.

The upper volume 14 comprises an inlet 16. The latter is arranged near to cover 8. It is designed to supply the upper volume 14 for example, by means of a pipe, with a culture medium containing microalgae and is preferably as distant from the passive filtering unit 10 as possible. The distance from inlet 16 to the passive filtering unit defines a height referred to as the effective height and designated by HU in FIG. 1.

By way of on-limiting illustration only, the distance between cover 8 and the center of inlet 16 is, for example, approximately 10 to 20 cm. This distance can also be estimated, still by way of non-limiting illustration, to range between one and three times the diameter of inlet 16.

The upper volume 14 also comprises an outlet 18. The latter is arranged near the passive filtering unit 10, preferably as close to the passive filtering unit 10 as possible. In all cases it is at a smaller height than inlet 16.

By way of non-limiting illustration only, the distance between the passive filtering unit 10 and the center of outlet 18 is, for example, approximately 5 to 15 cm. This distance can also be estimated, still by way of non-limiting illustration, to range between one and two times the diameter of outlet 18.

It can be noted that a pipe 20 comprising two bends is connected to outlet 18 of the upper volume 14 of concentrator 2. This pipe 20 has, in its mounted position shown in the drawings, a first horizontal portion 22 connected to outlet 18, a vertical section 24 connected to the first horizontal portion 22 by a first bend 26 and to a second horizontal portion 28 by a second bend 30. The horizontal position of the second section 28 relative to the passive filtering unit 10 defines a height referred to as the outlet height and designated by HS in FIG. 1. For each concentrator 2, the outlet height is less than the effective height HU.

Cover 8 closes the upper volume 14. It is for example formed by a membrane which is semi-permeable to oxygen (O₂) and is mounted so that oxygen can escape from the upper volume 14. Alternatively, the membrane (which is optional) can be placed in cover 8 so as to be at the highest point of concentrator 2.

Lower volume 12 comprises an outlet 32 which is preferably arranged in the lower part of concentrator 2, adjacent bottom 6. This outlet 32 is connected to a discharge pipe 34, which is preferably provided with a solenoid valve 36.

Lower volume 12 is intended to collect the filtrate obtained when a culture medium is introduced into upper volume 14. This filtrate is most often pure water. As shown in the Figures, the filtrate does not fill the entire lower volume 12 but only a portion of it, thus defining a level designated by N. The presence of solenoid valve 36 (and a level sensor not shown) maintains a level N in lower volume 12, which is substantially constant with time.

The space between the filtrate and the passive filtering unit 10 is preferably filled with carbon dioxide (CO₂), which is introduced into lower volume 12 through a nozzle 38. It is located for example, above the filtrate level, for example to three-quarters of the overall height of lower volume 12.

FIG. 1 therefore illustrates a concentrator 2 provided with an inlet 16 for the culture medium and with two outlets 18 and 32 separated by a passive filtering unit 10, the two outlets thus allowing gravity to act through two resultants and a culture medium to be obtained at the outlet 18, in which the microalgae concentration is higher than that of the culture medium fed through inlet 16.

FIG. 2 illustrates the combination of two concentrators, each corresponding to a concentrator as shown in FIG. 1 and described above.

In the preferred embodiment, concentrators 2 are arranged next to each other on a flat horizontal ground. The concentrators may be aligned as shown in FIG. 2 or may be arranged in a circle, form an L shape, etc.

It is preferable to use specific dimensions for each concentrator 2. Thus, in a preferred embodiment, each concentrator 2 has a different diameter and its own overall height. It may advantageously be provided that all passive filter units 10 are arranged at the same elevation or height (that is, at the same distance from the ground upon which concentrators 2 rest). It may then be provided that all outlets 32 corresponding to the lower volumes 12 of the concentrators, are also located at the same elevation or height. The same reasoning applies to the outlets 18 of the upper volumes of concentrators 2 and the injectors 38.

Concentrators 2 are connected together such that the second horizontal section 28 of each pipe 20 connected to an outlet 18 of a concentrator 2, referred to as the upstream concentrator, is connected to inlet 16 of one concentrator 2, referred to as the downstream concentrator. The first concentrator or most upstream concentrator, in turn, is fed directly with culture medium through its inlet 16, for example, from a photobioreactor, or other microalgae (culture) production means. Similarly, the most downstream concentrator 2 may include a pipe 20 as shown in FIG. 2, the latter, however, not being connected to a concentrator.

Concentrators 2 are designed and arranged relative to each other so that the inlet 16 of an upstream concentrator 2 is at an elevation (the distance from the ground on which it rests, in the case where all concentrators are arranged on the same horizontal plane, or otherwise, the distance with respect to a common horizontal reference plane) which is greater than that of the inlet of the corresponding downstream concentrator 2. Under the above assumption, according to which the passive filtering units 10 are all arranged at the same height, effective heights HU and outlet heights HS, which decrease from an upstream concentrator to a downstream concentrator, are then defined. The height difference ΔH=HU−HS for each concentrator is specific, and is adapted to the desired characteristics of the device for harvesting microalgae.

FIG. 3 illustrates a passive filtering unit 10 in an exploded perspective view. This passive filtering unit 10 includes, in the preferred embodiment shown here, eight layers arranged symmetrically with respect to the center of said passive filtering unit 10. In this embodiment, each layer of the passive filtering unit is disc shaped.

Arranged in order from the outside to the inside of the passive filtering unit 10 shown in FIG. 3, are:
a wire mesh 40 with square meshes,
a first filter membrane 42 with a 1.2 μm (1.2 10⁻⁶ m) porosity,
a second filter membrane 44 with a 0.8 μm (0.8 10⁻⁶ m) porosity, and
a third filter membrane 46 with a 0.4 μm (0.4 10⁻⁶ m) porosity.

Of course, the porosity of the filter membranes is given by way of non-limiting example. This porosity can vary depending on the size of the microorganisms in the culture medium. In addition, the described passive filtering unit comprises eight layers. Depending on the concentration to be achieved and the type of microorganisms, the number and/or nature and/or thickness of the layers can also be adapted.

The structure of the wire mesh can also be adapted according to the treated medium.

The original structure for a passive filtering unit is particularly well suited to the present invention, but could also be used in other applications within a concentration device.

Such a passive filtering unit 10 can in particular provide fine CO₂ bubbles after passing through the passive filtering unit 10, thus preventing any sedimentation of microalgae (or other organisms) in the upper volume of the concentrator. Additionally, to clean such a passive filtering unit 10, it is sufficient to turn it around so that in-operation self-cleaning of the passive filtering unit can be achieved.

The different layers of the passive filtering unit 10 are surrounded, for example, by a circular sealing joint, not shown, made of a resilient material. This joint could be arranged so as to furthermore surround the outer surface of each wire mesh 40 in order to maintain consistency between the layers of the passive filtering unit before it is mounted into a concentrator 2.

The device for harvesting microalgae shown in FIG. 2 may then operate as explained below. This device is intended to operate continuously and by gravity.

A culture medium obtained from a photobioreactor is supplied to the first concentrator, or most upstream concentrator. For purely illustrative purposes, it is assumed here that the concentration of algae in the culture medium is 4 g/l of dry matter. The height of the inlet 16 of the first concentrator 2 is, for example, approximately 5 m above the ground on which this concentrator 2 (and the next ones) rests.

Here and thereafter a steady state operation of the device is assumed (rather than the start of the harvesting process).

The upper volume 14 of the first concentrator 2, and of the following ones, is entirely filled with the culture medium. Cover 8, which is formed by a membrane that is semi-permeable to oxygen, or which comprises such a membrane, allows oxygen released from the culture medium to exit the concentrator.

The culture medium exerts a pressure on the passive filtering unit 10, which allows gravity to act through two resultants (outlets of the upper and lower volumes). Each concentrator may have a diameter different from that of the other concentrators, although the passive filtering units 10, which preferably have the same structure, each have dimensions (areas) adapted to the concentrator 2 in which they are provided. The culture medium is then concentrated, and pure (or at least microalgae-free) water passes through the passive filtering unit 10 and reaches the lower volume 12 of the concentrator. As already mentioned, the water level N is kept constant in each concentrator 2, thanks in particular to the presence of a valve 36 on each discharge pipe 34.

The presence of carbon dioxide (CO₂) in lower volume 12 of each concentrator 2 maintains a constant pressure in lower volume 12. Slow diffusion of carbon dioxide through the passive filtering unit 10 serves to slowly drain the microalgae present in upper volume 14 towards the corresponding outlets 18 and prevents microalgae sedimentation in upper volume 14.

In each concentrator 2, an inlet flow rate DE1 of culture medium, an outlet flow rate DS1 of pure water through the discharge pipe 34, and an outlet flow rate DS2 of culture medium through pipe 20, are observed. Here, only liquid flow rates, rather than the gas flow rates related in particular to the introduction of carbon dioxide into the system, are considered. This then leads to the following equation:

DE1=DS1+DS2

The various dimensional parameters (in particular the outlet heights and diameter) may be adapted so that, for example, DS1=DS2. Solenoid valves (not shown) may also be used in pipe 20 for better management of the microalgae concentration process. In this case, given by way of illustrative example, a microalgae concentration in the downstream concentrator is then observed, which is twice that of the culture medium in the upstream concentrator. The microalgae concentration therefore doubles from an upstream concentrator to a downstream concentrator. As a result, the viscosity of the medium varies. The dimensions of pipe 20, particularly its inner diameter and the radius of curvature of bends 26, 30, are advantageously adapted to each stage of the harvesting device according to the invention.

Returning to the assumption of a culture medium having an starting concentration of 4 g/l at the inlet 16 of the first concentrator 2, this concentration is 8 g/l at the outlet 18 of the same concentrator and, in the case illustrated in FIG. 2, where seven concentrators are arranged in series one after the other, the concentration at the outlet of the device (that is, at the outlet of the last concentrator) is 512 g/l.

Based on these numerical assumptions, if the inlet flow rate of the system (with seven concentrators 2) is 10,0001/h (or 10 m³/h) with an algal medium concentration of 4 g/l, the outlet flow rate in the most downstream pipe 20 will be 78 l/h, with a concentration of 512 g/l of dry matter. Additionally, 9,922 l of pure water will be available. This water can be recycled to supply photobioreactors with water (requiring pumping) or for other uses.

Here, the microalgae concentration process is only carried out by gravity and continuously. The system is preferably hermetically sealed to avoid contamination of the algal medium throughout the concentration process. However, the use of concentrators without a cover can be envisaged. Such a system is more difficult to manage because of the risk of overflow.

Of course, it is possible to adjust the number of concentrators according to the inlet concentration of the culture medium and/or the desired outlet concentration.

The present invention is not limited to the preferred embodiment described above by way of non-limiting example and illustrated in the drawings, and to the discussed alternatives. It covers all embodiments within the ability of those skilled in the art and in the scope of the appended claims.

1. A method for harvesting microalgae in a culture medium, characterized in that the culture medium passes through a set of consecutive tanks, referred to as concentrators (2), thereby defining a series of upstream and downstream concentrators, in that each concentrator (2) comprises, on the one hand, a casing which defines an inner space and, on the other hand, a passive filtering unit (10) which separates said inner space into an upper volume (14) and a lower volume (12), in that the upper volume (14) comprises an inlet (16) for introducing a culture medium into said upper volume (14) and an outlet (18) arranged at a lower position with respect to the inlet (16) for extracting a culture medium having a higher microalgae concentration from the upper volume (14), in that the lower volume (12) comprises an outlet (32) for extracting a liquid from the lower volume (12), in that the inlet (16) of the upper volume (14) of a downstream concentrator is connected to the outlet (18) of the upper volume (14) of the upstream concentrator, and in that a concentrated culture medium is harvested at the outlet (18) of the upper volume (14) of a concentrator (2).

2. The method for harvesting microalgae according to claim 1, characterized in that the inlet (16) of the upper volume (14) of an upstream concentrator is located at a greater height than the inlet (16) of the upper volume (14) of the downstream concentrator.

3. The method for harvesting microalgae according to claim 1, characterized in that carbon dioxide is injected into the lower volume (12) of at least one concentrator (2).

4. A device for harvesting microalgae, characterized in that it comprises a set of consecutive tanks, referred to as concentrators (2), thereby defining a series of upstream and downstream concentrators, in that each concentrator (2) has, on the one hand, a casing which defines an inner space and, on the other hand, a passive filtering unit (10) which separates said inner space into an upper volume (14) and a lower volume (12), in that the upper volume (14) comprises an inlet (16) for introducing a culture medium into said upper volume (14) and an outlet (18) arranged at a lower position with respect to the inlet (16) for extracting a culture medium having a higher microalgae concentration from the upper volume (14), in that the lower volume (12) comprises an outlet (32) for extracting a liquid from the lower volume (12), and in that the inlet (16) of the upper volume (14) of a downstream concentrator is connected to the outlet (18) of the upper volume (14) of the upstream concentrator.

5. The device according to claim 4, characterized in that the inlet (16) of the upper volume (14) of an upstream concentrator is located at a greater height than the inlet (16) of the upper volume (14) of the downstream concentrator.

6. The device according to claim 4, characterized in that the lower volume (12) of at least one concentrator comprises means (38) for injecting a pressurized gas into said lower volume (12).

7. The device according to claim 5, characterized in that all concentrators (2) are placed in the same plane.

8. The device according to claim 7, characterized in that all passive filtering units (10) of the device are arranged at the same height.

9. The device according to claim 4, characterized in that the upper volume (14) of at least one concentrator (2) has in its upper portion a membrane which is semi-permeable to oxygen and/or a membrane which is semi-permeable to hydrogen, allowing oxygen and/or hydrogen to exit the concentrator.

10. The device according to claim 4, characterized in that a passive filtering unit (10) of one concentrator (2) comprises two wire meshes (40) between which filter membranes (42, 44, 46) are located, whose porosity decreases from one wire mesh (40) to the center of the passive filtering unit (10).

11. The method for harvesting microalgae according to claim 2, characterized in that carbon dioxide is injected into the lower volume (12) of at least one concentrator (2).

12. The device according to claim 5, characterized in that the lower volume (12) of at least one concentrator comprises means (38) for injecting a pressurized gas into said lower volume (12).

13. The device according to claim 6, characterized in that all concentrators (2) are placed in the same plane.

14. The device according to claim 5, characterized in that the upper volume (14) of at least one concentrator (2) has in its upper portion a membrane which is semi-permeable to oxygen and/or a membrane which is semi-permeable to hydrogen, allowing oxygen and/or hydrogen to exit the concentrator.

15. The device according to claim 5, characterized in that a passive filtering unit (10) of one concentrator (2) comprises two wire meshes (40) between which filter membranes (42, 44, 46) are located, whose porosity decreases from one wire mesh (40) to the center of the passive filtering unit (10).
